(19) United States
(12) Patent Application Publication
(10) Pub. No.: US 2023/0168161 A1
(43) Pub. Date: Jun. 1, 2023

(54) AUTOMATIC PREPROCESSING APPARATUS FOR BIOPSY

(71) Applicant: Vectornate USA Inc, (US)

(72) Inventors: Sean Taesun Yeom, Jeollanamdo (KR); Ji-Hyun Shin, Jeollanam-do (KR)

(21) Appl. No.: 17/974,555

(22) Filed: Oct. 27, 2022

Related U.S. Application Data
(60) Provisional application No. 63/237,528, filed on Aug. 27, 2021.

(51) Int. Cl. G01N 1/31; A61B 10/00; G01N 35/10

(52) U.S. Cl. CPC G01N 1/312; A61B 10/0096; G01N 35/1065; G01N 2035/103

(57) ABSTRACT

The present invention relates to an automatic preprocessing apparatus for biopsy, comprising: a housing having an inner space; an inspection module provided in the inner space of the housing; and a sliding door provided with a storage space, and slidably coupled to the housing so as to be inserted into the inner space of the housing or to protrude to the outside of the housing, wherein the inspection module is movable in X-axis, Y-axis, and Z-axis directions, and comprises a moving part capable of gripping an inspection tool.

### CROSS-REFERENCE TO RELATED APPLICATION

This application claims priority to and the benefit of U.S. Patent Provisional Application No. 63/237,528, filed on Aug. 27, 2021, the disclosure of which is incorporated herein by reference in its entirety.

### BACKGROUND

### Technical Field

The present invention relates to an automatic preprocessing apparatus for biopsy and, more specifically, to an automatic preprocessing apparatus for biopsy comprising: an inspection module for performing a biopsy while automatically moving an inspection tool required for biopsy; and a sliding door sliding to the outside of a housing while storing the inspection tool.

### Description of the Related Art

In order to diagnose a disease occurring in a living body, a part of tissue is collected from the living body, and a biopsy is performed through the collected tissue. Conventional biopsies were performed while an inspector used an inspection tool.

However, the conventional biopsy apparatus has the following problems. When an inspector performs a biopsy using an inspection tool, there is a problem in that contamination occurs in the inspection tool during the use of the inspection tool.

In order to solve such a problem, an automatic biopsy apparatus is introduced. However, the conventional automatic biopsy apparatus has a problem in that it is difficult to easily insert a tool required for a biopsy into the apparatus.

In addition, various inspection tools such as a tube, a pipette, a slide, and the like are used in the biopsy, and the conventional automatic biopsy apparatus has a problem in that it is difficult to put or move the various tools in an appropriate place.

In addition, the conventional automatic biopsy apparatus has a problem in that it is difficult to process an inspection tool that has been inspected, and an inspector is infected by the inspection tool that has been inspected.

In particular, when pathogens existing in a living body are detected, there is a problem in that an inspector is frequently infected by pathogens during a process of processing an inspection tool.

### SUMMARY

### Technical Problem

The present invention has been made in an effort to solve the above-described problems, and more particularly, to an automatic preprocessing apparatus for biopsy, which includes an inspection module for performing a biopsy while automatically moving an inspection tool required for a biopsy, and a sliding door sliding to the outside of a housing while storing the inspection tool.

### Technical Solution

The automatic preprocessing apparatus for biopsy of the present invention to solve the above-described problems comprises: a housing having an inner space; an inspection module provided in the inner space of the housing; and a sliding door provided with a storage space and slidably coupled to the housing so as to be inserted into the inner space of the housing or to protrude to the outside of the housing, wherein the inspection module is movable in X-axis, Y-axis, and Z-axis directions, and includes a moving part capable of gripping an inspection tool.

The moving part of the automatic preprocessing apparatus for biopsy of the present invention to solve the above-described problems may include a first moving part capable of gripping a tube and a second moving part capable of gripping a pipette.

The first moving part of the automatic preprocessing apparatus for biopsy of the present invention to solve the above-described problems may grip the cap of the tube through adsorption.

The inspection module of the automatic preprocessing apparatus for biopsy of the present invention to solve the above-described problems may include a seating part on which an inspection tool is seated.

The inspection module of the automatic preprocessing apparatus for biopsy of the present invention to solve the above-described problems may include a cap adjustment part for removing a cap from an upper portion of a tube seated on the seating part or coupling the cap to the upper portion of the tube seated on the seating part.

The cap adjustment part of the automatic preprocessing apparatus for biopsy of the present invention to solve the above-described problems may include a plurality of contact parts which are slidable in a direction away from a side surface of the cap, and a rotation part which rotates the contact parts while the contact parts are coupled, and an elastic body may be coupled to an end of the contact parts.

The inspection module of the automatic preprocessing apparatus for biopsy of the present invention to solve the above-described problems may include a material injection part for injecting a material into a tube seated on the seating part.

The inspection module of the automatic preprocessing apparatus for biopsy of the present invention to solve the above-described problems may include a mixing part capable of applying vibration to an inspection tool or rotating the inspection tool.

The inspection module of the automatic preprocessing apparatus for biopsy of the present invention to solve the above-described problems may include an information recognition part which is movable in X-axis, Y-axis, and Z-axis and can recognize a code displayed on an inspection tool.

The housing of the automatic preprocessing apparatus for biopsy of the present invention to solve the above-described problems may include a display part for displaying information recognized by the information recognition part to the outside.

The code formed in the inspection tool of the automatic preprocessing apparatus for biopsy of the present invention to solve the above-described problems may be a QR code.

An inspection tool may be stored in the storage space of the sliding door of the automatic preprocessing apparatus for biopsy of the present invention to solve the above-described problems.

At least one of a tube storage part in which a tube is stored, a pipette storage part in which a pipette is stored, and a slide storage part in which a slide for fixing a specimen is stored may be provided in the storage space of the sliding door of the automatic preprocessing apparatus for biopsy of the present invention to solve the above-described problems.

The automatic preprocessing apparatus for biopsy of the present invention to solve the above-described problems further comprises a waste processing part which is provided with an outlet communicating with the inner space of the housing and a discharge bag capable of storing an inspection tool therein, and the moving part can move the used inspection tool to the discharge bag through the outlet.

The waste processing part of the automatic preprocessing apparatus for biopsy of the present invention to solve the above-described problems may include a sealing part capable of sealing an upper portion of the discharge bag in which the used inspection tool is stored.

The moving part of the automatic preprocessing apparatus for biopsy of the present invention to solve the above-described problems may include a second moving part capable of gripping a pipette, and a separation part having a groove smaller than an upper diameter of the pipette is provided at an upper portion of the outlet, and the second moving part may dispose the pipette at a lower portion of the groove and separate the pipette from the second moving part while moving the pipette upward, thereby lowering the pipette to the outlet.

### BRIEF DESCRIPTION OF THE DRAWINGS

FIG. 1 is a diagram illustrating an automatic preprocessing apparatus for biopsy according to an embodiment of the present invention.

FIG. 2 is a view illustrating that a sliding door protrudes to the outside of a housing according to an embodiment of the present invention.

FIG. 3 is a view illustrating that an inspection tool is stored in a storage space of a sliding door according to an embodiment of the present invention.

FIG. 4 is a view illustrating an inner space of a housing according to an exemplary embodiment of the present invention.

FIG. 5 is a view illustrating a first moving part and a second moving part according to an embodiment of the present invention.

FIG. 6 and FIG. 7 are views illustrating a process of removing a cap of a tube through a cap adjustment part according to an embodiment of the present invention.

FIG. 8 is a view illustrating injecting a material into a tube through a material injection part according to an embodiment of the present invention.

FIG. 9 is a diagram illustrating a mixing part according to an embodiment of the present invention.

FIG. 10 and FIG. 11 are views illustrating moving a specimen from a first tube to a second tube through a pipette according to an embodiment of the present invention.

FIG. 12 is a view illustrating moving a specimen to a slide through a pipette according to an embodiment of the present invention.

FIG. 13 is a view illustrating a display part according to an embodiment of the present invention.

FIG. 14 is a view illustrating removing a pipette coupled to a second moving part through a separation part according to an embodiment of the present invention.

FIG. 15 is a view illustrating a discharge bag and a sealing part of a waste processing part according to an embodiment of the present invention.

### DETAILED DESCRIPTION

In the present specification, the principle of the present invention will be described and embodiments will be disclosed so that the scope of the present invention can be clarified and those skilled in the art can implement the present invention. The disclosed embodiments may be implemented in various forms.

Expressions such as “include” or “may include” that may be used in various embodiments of the present invention indicate the presence of corresponding functions, operations, or components that have been disclosed, and do not limit one or more additional functions, operations, or components. In addition, in various embodiments of the present invention, it should be understood that the terms “include” or “have” are intended to designate that there is a feature, a number, a step, an operation, a component, a part, or a combination thereof described in the specification, but do not preclude the possibility of the presence or addition of one or more other features, numbers, steps, operations, components, parts, or combinations thereof.

When it is stated that a component is “connected and coupled” to another component, it should be understood that while the component may be directly connected or coupled to the other component, there may be a new other component between the component and the other component. On the other hand, when it is stated that a component is “directly connected” or “directly coupled” to another component, it should be understood that there is no new component between the component and the other component.

While terms such as “first” and “second” used herein may be used to describe various components, the components should not be limited by the terms. The terms are used only for the purpose of distinguishing one component from another component.

The present invention relates to an automatic preprocessing apparatus for biopsy, and relates to an automatic preprocessing apparatus for biopsy comprising: an inspection module for conducting an inspection while automatically moving an inspection tool required for biopsy; and a sliding door sliding to the outside of a housing while storing the inspection tool. Hereinafter, preferred embodiments of the present invention will be described in detail with reference to the accompanying drawings.

The automatic preprocessing apparatus for biopsy according to an embodiment of the present invention includes a housing 110, an inspection module 200, and a sliding door 120.

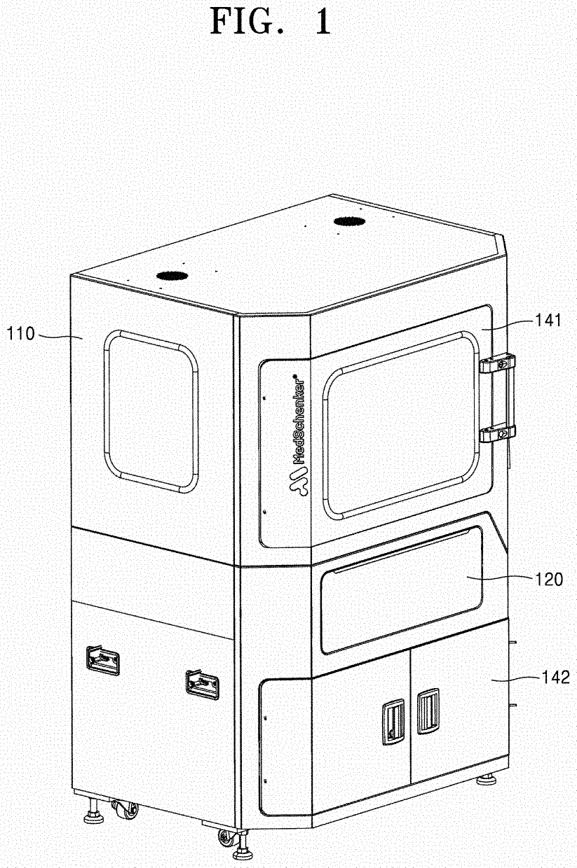

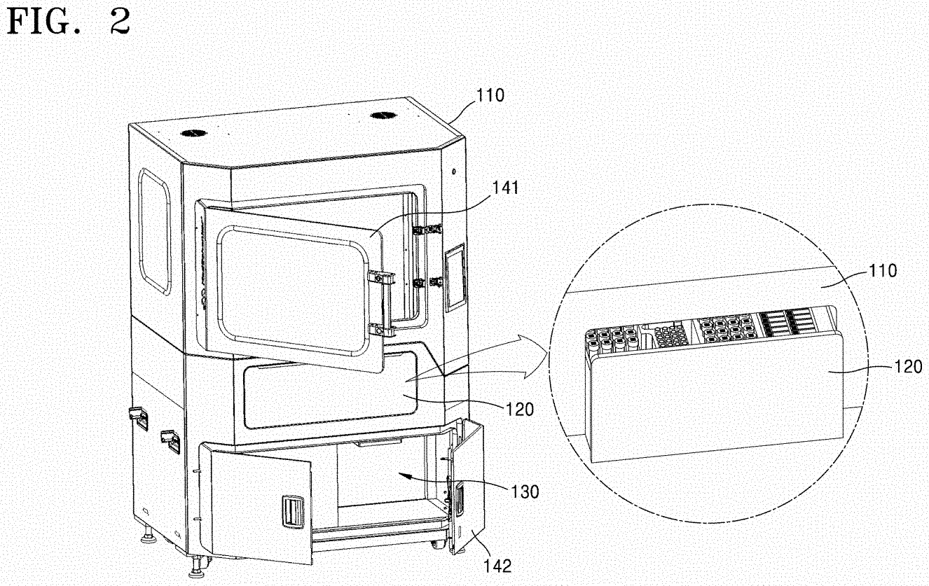

Referring to FIGS. 1 and 2, the housing 110 has an inner space, and the housing 110 may be a frame of an automatic preprocessing apparatus for biopsy.

The inspection module 200 is provided in the inner space of the housing 110. The inspection module 200 is movable in X-axis, Y-axis, and Z-axis and includes a moving part 210 capable of gripping the inspection tool.

The moving part 210 may move in the inner space of the housing 110. The moving part 210 may move the inspection tool from a designated first point to a designated second point in the inner space of the housing 110.

According to an embodiment of the present invention, the inspection tool may include various tools for performing a biopsy. The inspection tool may include a tube 10 having a cap 20, a pipette 30, and a slide 40, and may also include various tools for performing a biopsy.

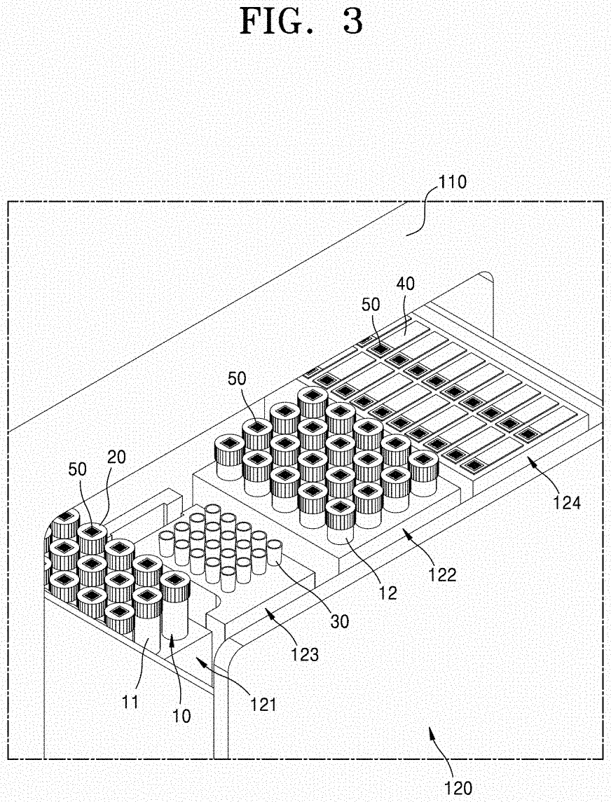

Referring to FIGS. 2 and 3, the sliding door 120 may have a storage space, and may be inserted into an inner space of the housing 110 or protrude to the outside of the housing 110.

The sliding door 120 may be a door provided in the housing 110. The sliding door 120 may be inserted into the inner space of the housing 110 as shown in FIG. 1, and may protrude to the outside of the housing 110 as shown in FIG. 2 through a sliding movement.

According to an embodiment of the present invention, various apparatus may be provided in the sliding door 120 and the housing 110 to insert the sliding door 120 into the inner space of the housing 110 or protrude to the outside of the housing 110.

According to an embodiment of the present invention, the sliding door 120 may be operated in a touch manner. Specifically, when the sliding door 120 is touched in the state of FIG. 1, the sliding door 120 may protrude to the outside of the housing 110 as shown in FIG. 2.

Thereafter, when a force is applied to the sliding door 120 protruding to the outside of the housing 110, the sliding door 120 may slide and be inserted into the inner space of the housing 110 as shown in FIG. 1.

However, the sliding door 120 is not limited to being operated in a touch manner, and may be operated in various methods as long as the sliding door 120 is inserted into the inner space of the housing 110 or protrudes to the outside of the housing 110.

Referring to FIG. 3, an inspection tool may be stored in a storage space of the sliding door 120. Various inspection tools may be stored in the storage space of the sliding door 120, and a plurality of inspection tools may be stored in the storage space of the sliding door 120 in a modular manner.

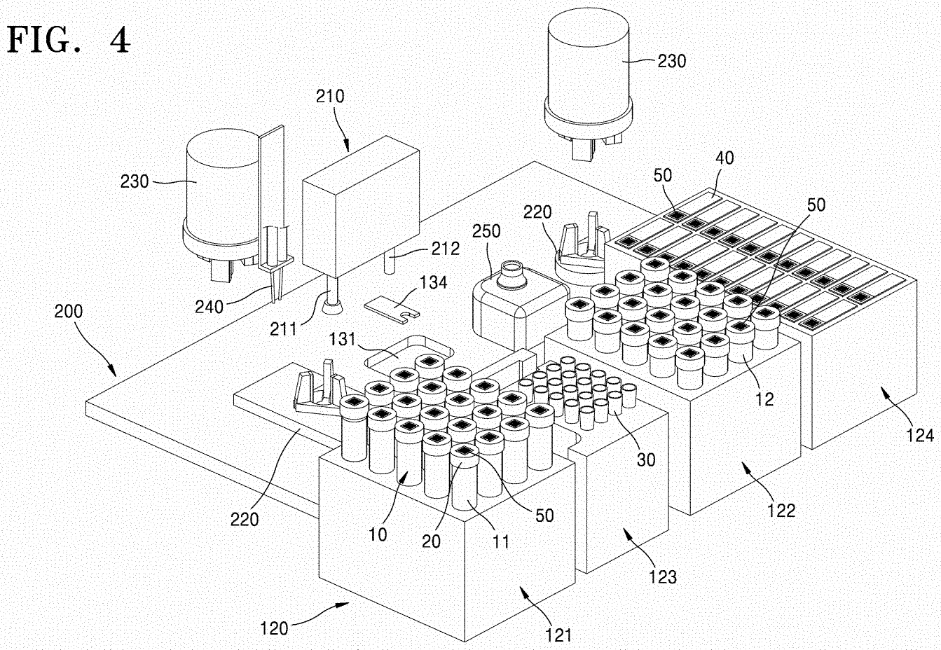

Referring to FIG. 4, when the sliding door 120 is inserted into the inner space of the housing 110, the storage space of the sliding door 120 and the inner space of the housing 110 may communicate with each other.

The storage space of the sliding door 120 is connected to the inner space of the housing 110 in which the inspection module 200 is provided, and the inspection module 200 may grip the inspection tool stored in the storage space of the sliding door 120 through the moving part 210.

As described above, when the sliding door 120 which is slidable is provided in the housing 110 and the storage space of the sliding door 120 and the inner space of the housing 110 in which the inspection module 200 is provided are communicated with each other, the inspection tool may be easily supplied into the housing 110.

Specifically, the inspector may protrude the sliding door 120 to the outside of the housing 110 in order to insert the inspection tool into the inner space of the housing 110. The inspector may store the inspection tool in the storage space of the sliding door 120 and supply the inspection tool into the inner space of the housing 110 while inserting the sliding door 120 into the inner space of the housing 110.

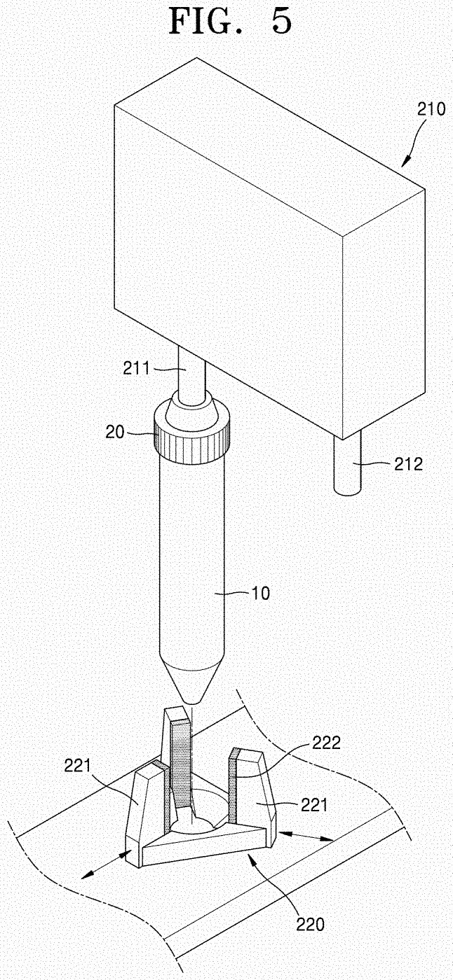

Referring to FIGS. 4 and 5, the moving part 210 of the inspection module 200 includes a first moving part 211 capable of gripping the tube 10 and a second moving part 212 capable of gripping the pipette 30.

The first moving part 211 and the second moving part 212 may be movable in X-axis, Y-axis, and Z-axis directions, and the tube 10 and the pipette 30 may be moved to a designated place through the first moving part 211 and the second moving part 212.

The first moving part 211 is provided to move the tube 10 by gripping the tube 10, and the first moving part 211 may grip the cap 20 of the tube 10 through adsorption. Specifically, the end of the first moving part 211 may be formed in a tube shape that is open to the outside, and an adsorption force that sucks air may be generated at the end of the first moving part 211.

An end of the first moving part 211 may be in contact with the cap 20 of the tube 10. When the end of the first moving part 211 is in contact with the cap 20 of the tube 10, the tube 10 may be moved through the end of the first moving part 211 by an adsorption force.

The second moving part 212 is configured to grip and move the pipette 30, and the second moving part 212 includes a moving bar inserted into one side of the pipette 30. The moving bar may have a diameter larger than an inner diameter of the pipette 30 and may be press-fitted into the pipette 30. When the moving bar of the second moving part 212 is inserted into the pipette 30, the pipette 30 may be moved through the second moving part 212.

According to an embodiment of the present invention, the moving part 210 may be provided with another type of moving part capable of gripping other inspection tools than the tube 10 and the pipette 30.

Referring to FIG. 5, the inspection module 200 may include a seating part 220 on which the inspection tool is seated. The seating part 220 may be seated while the inspection tool is fixed, and the inspection tool may be seated on the seating part 220 through the moving part 210.

Referring to FIG. 5, the seating part 220 includes a fixing part 221 which is slidable in a direction toward or away from a side surface of the inspection tool.

The fixing part 221 may be formed in a bar shape, and a plurality of fixing parts 221 may be provided. When the inspection tool is seated on the seating part 220, the fixing part 221 which is slidable in a direction away from a side surface of the inspection tool.

When the inspection tool is seated on the seating part 220, the fixing part 221 is brought into contact with the side surface of the inspection tool while sliding in a direction toward the side surface of the inspection tool. As the fixing part 221 slides to contact the side surface of the inspection tool, the inspection tool may be fixed to the seating part 220.

An elastic body 222 may be coupled to an end of the fixing part 221. The elastic body 222 may be made of an elastic material, and the elastic body 222 is a portion directly contacting a side surface of the inspection tool.

When the fixing part 221 is in contact with the side surface of the inspection tool through the sliding movement of the fixing part 221, the inspection tool may be damaged by the fixing part 221. In order to prevent this, the elastic body 222 may be provided at a portion where the fixing part 221 directly contacts the side surface of the inspection tool.

According to an embodiment of the present invention, the tube 10 may be moved through the first moving part 211 to be seated on the seating part 220. When the tube 10 is seated on the seating part 220, the fixing part 221 is slidable in a direction away from the side surface of the inspection tool.

When the tube 10 is seated on the seating part 220, the fixing part 221 is brought into contact with the side surface of the tube 10 while sliding in a direction toward the side surface of the inspection tool. As the fixing part 221 slides to contact the side surface of the tube 10, the tube 10 seated on the seating part 220 may be fixed.

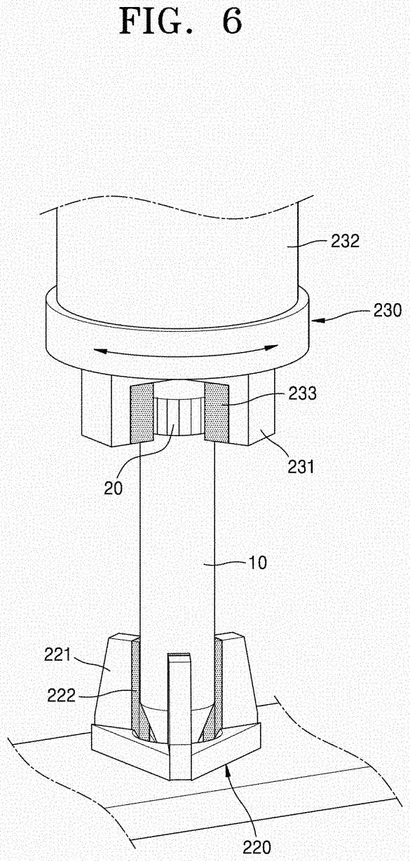

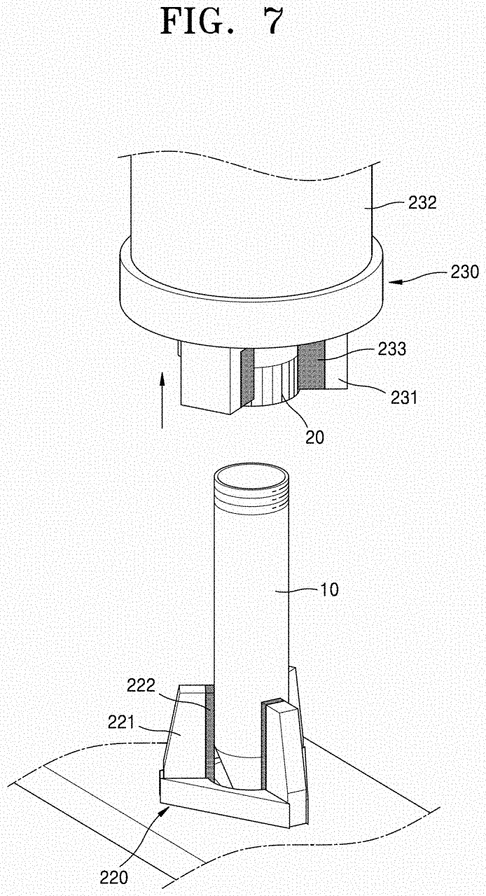

Referring to FIGS. 6 and 7, the inspection module 200 may include a cap adjustment part 230 capable of removing the cap 20 from an upper portion of the tube 10 seated on the seating part 220 or coupling the cap 20 to the upper portion of the tube seated on the seating part 220.

The cap adjustment part 230 may automatically remove the cap 20 from the tube 10. The cap adjustment part 230 includes a contact part 231 and a rotation part 232.

The contact part 231 may be slidable in a direction toward or away from a side surface of the cap 20. The contact part 231 may be formed in a bar shape, and a plurality of contact parts 231 may be provided.

The rotation part 232 rotates the contact part 231 while the contact part 231 is coupled thereto. A plurality of contact parts 231 may be coupled to a side surface of the rotation part 232. The rotation part 232 may be rotated in place. When the rotation part 232 rotates, the plurality of contact parts 231 also rotate.

When the tube 10 is seated on the seating part 220, the contact part 231 may be in contact with the side surface of the cap 20 while sliding in a direction toward the side surface of the cap 20 of the tube 10.

When the plurality of contact parts 231 contact the side surface of the cap 20, the plurality of contact parts 231 are rotated through the rotation part 232 to separate the cap 20 from the tube 10. Thereafter, when the cap 20 is again coupled to the tube 10, the cap 20 may be coupled to the tube 10 while rotating the rotation part 232 in the opposite direction.

An elastic body 233 may be coupled to an end of the contact part 231. The elastic body 233 may be made of an elastic material, and the elastic body 233 is a portion directly contacting a side surface of the cap 20.

When the contact part 231 contacts the side surface of the cap 20 through the sliding movement of the contact part 231, the cap 20 may be damaged by the contact part 231. To prevent this, the elastic body 233 may be provided at a portion where the contact part 231 directly contacts the side surface of the cap 20.

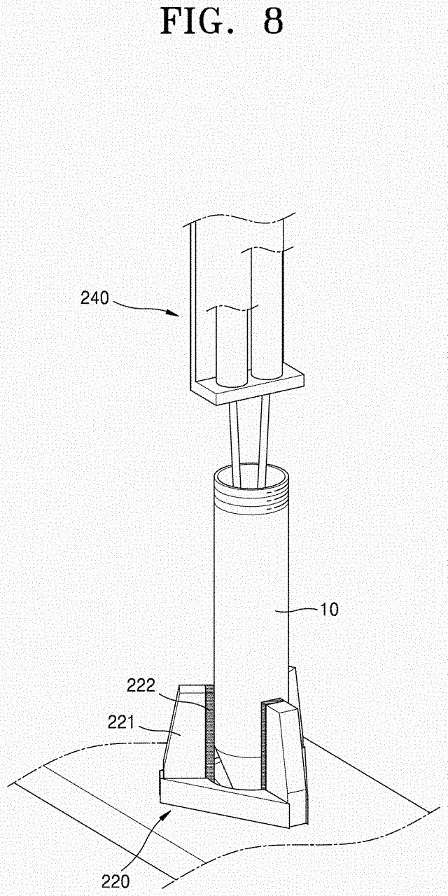

Referring to FIG. 8, the inspection module 200 may include a material injection part 240 capable of injecting a material into the tube 10 seated on the seating part 220. The material injection part 240 may include a needle and a connection pipe.

After the tube 10 is seated on the seating part 220 through the first moving part 211, the cap 20 provided on an upper portion of the tube 10 may be separated through the cap adjustment part 230. After the cap 20 is separated from the tube 10, a material may be injected into the tube 10 through the material injection part 240.

According to an embodiment of the present invention, the material injected into the tube 10 through the material injection part 240 may be N-acetyl-L-cysteine NaOH (NALC NaOH), which is a pretreatment solution for increasing the discrimination of specimen. NALC NaOH (N-acetyl-L-cysteine NaOH) may sterilize bacteria in the specimen and homogenize mucilage components. In addition, it is possible to make the mucus component of the specimen together with water and to easily dye Mycobacterium tuberculosis.

However, the material injected into the tube 10 through the material injection part 240 is not limited thereto. The material injected into the tube 10 through the material injection part 240 may be a solution, and the material injected into the tube 10 through the material injection part 240 may be various materials required for biopsy.

After the material is injected into the tube 10 through the material injection part 240, the cap 20 may be coupled to an upper portion of the tube 10 through the cap adjustment part 230. In this case, the rotation part 232 of the cap adjustment part 230 may rotate in a direction opposite to the direction in which the cap 20 is separated.

After the cap 20 is coupled to the tube 10 through the cap adjustment part 230, the contact part 231 is slidable in a direction away from the side surface of the cap 20.

The seating part 220, the cap adjustment part 230, and the material injection part 240 may be movable in X-axis, Y-axis, and Z-axis directions in the inner space of the housing 110.

In addition, according to an embodiment of the present invention, the seating part 220, the cap adjustment part 230, and the material injection part 240 may be moved only in one direction or may be fixed to a specific point.

According to an embodiment of the present invention, the cap adjustment part 230 and the material injection part 240 may be fixed to a specific point of the inner space of the housing 110, and the seating part 220 may be moved in a direction in which the cap adjustment part 230 and the material injection part 240 are provided.

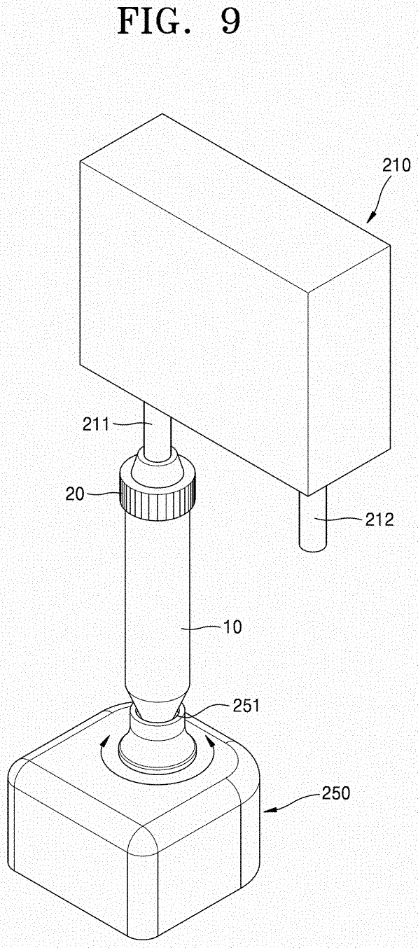

Referring to FIG. 9, the inspection module 200 may include a mixing part 250 for applying vibration to the inspection tool or rotating the inspection tool. The mixing part 250 may be provided with a groove 251 into which the inspection tool may be inserted.

According to an embodiment of the present invention, after injecting a material into the tube 10 through the material injection part 240, the tube 10 may be moved to the mixing part 250. In this case, the tube 10 may be moved from the seating part 220 to the mixing part 250 through the first moving part 211.

The tube 10 may be inserted into the groove 251 provided in the mixing part 250, and vibration may be applied to the tube 10 or the tube 10 may be rotated through the mixing part 250. Through this, the material injected into the tube 10 may be mixed.

The inspection module 200 according to an embodiment of the present invention may include a plurality of seating parts 220 and a plurality of cap adjustment parts 230. In addition, the tube 10 according to an embodiment of the present disclosure may include different types of tubes. Specifically, the tube 10 may include the first tube 11 and the second tube 12.

The first tube 11 may be a sample tube for moving or mixing materials, and the second tube 12 may be a tube for dyeing materials.

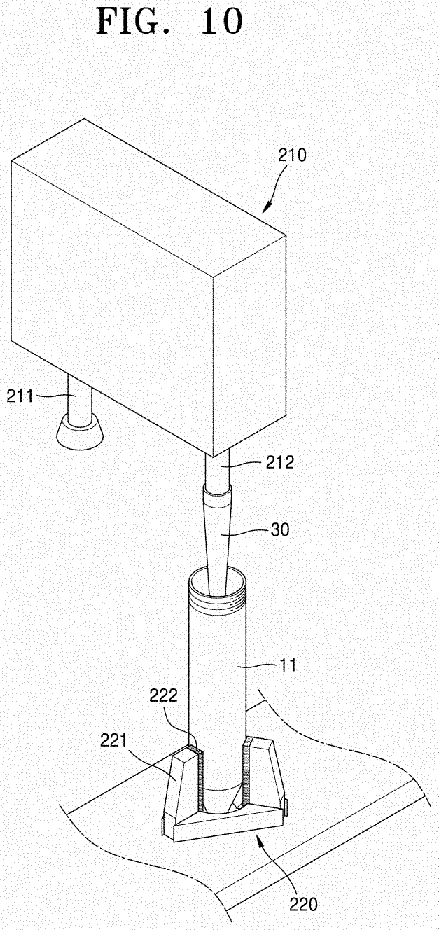

Referring to FIG. 10, the second moving part 212 may move the pipette 30, and may move a material from the first tube 11 to the second tube 12 through the pipette 30.

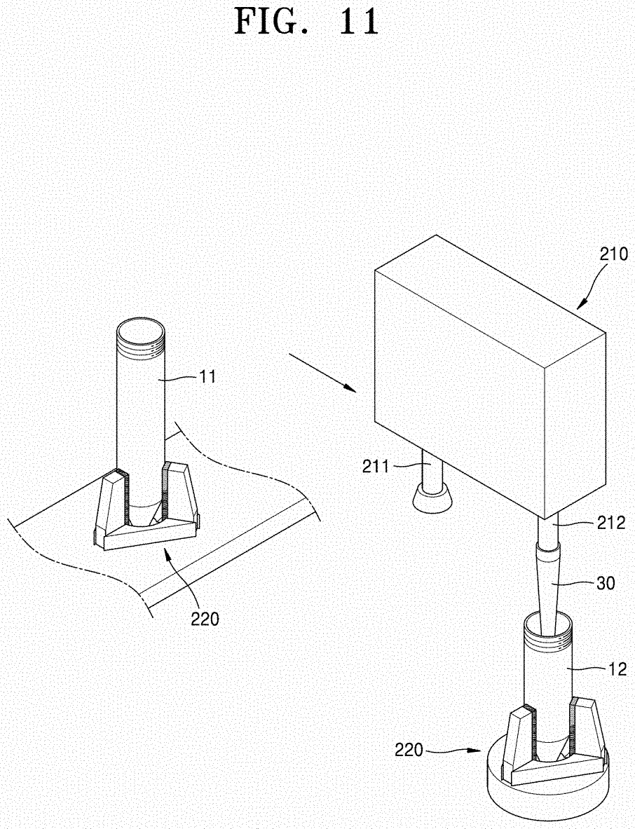

Referring to FIGS. 10 and 11, the first tube 11 is seated on the seating part 220 to remove the cap from an upper portion of the first tube 11 through the cap adjustment part 230. Thereafter, the second tube 12 is seated on the other seating part 220, and the cap is removed from an upper portion of the second tube 12 through the cap adjustment part 230.

The pipette 30 is moved to the first tube 11 through the second moving part 212 to collect the material of the first tube 11. Thereafter, the pipette 30 may be moved to the second tube 12 through the second moving part 212, so that the material collected by the pipette 30 may be moved to the second tube 12.

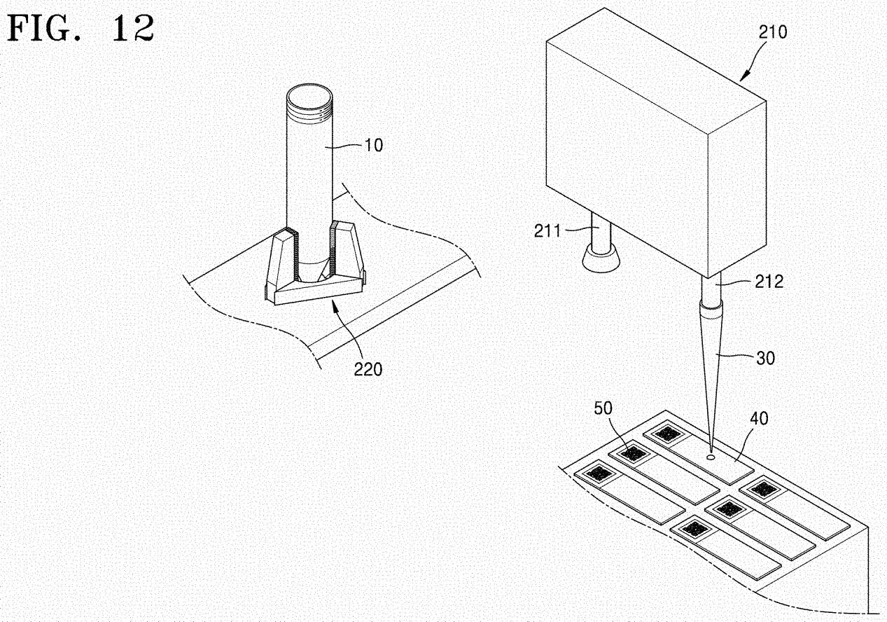

Referring to FIG. 12, a material may be directly coated on the slide 40 through the second moving part 212 and the pipette 30. The slide 40 is formed in a plate shape to fix the specimen, and an infrared lamp may be provided at a lower portion of the slide 40. The specimen coated on the slide 40 may be fixed through an infrared lamp.

An inspection tool may be stored in the storage space of the sliding door 120 according to an embodiment of the present disclosure, and various types of inspection tools may be stored in the storage space in a modular manner.

In detail, the storage space of the sliding door 120 may be provided with at least one of a tube storage part in which the tube 10 is stored, a pipette storage part 123 in which a pipette is stored, and a slide storage part 124 in which a slide for fixing a specimen is stored.

The tube storage part stores a plurality of tubes. Referring to FIG. 3, the tube storage part may include a first tube storage part 121 storing the plurality of first tubes 11 and a second tube storage part 122 storing the plurality of second tubes 12. The pipette storage part 123 stores a plurality of the pipettes 30, and the slide storage 124 stores a plurality of the slides 40.

As described above, the storage space of the sliding door 120 communicates with the inner space of the housing 110 in which the inspection module 200 is provided. The inspection module 200 may select and use an inspection tool stored in the storage space of the sliding door 120 through the moving part 210.

Although it has been described that the tube storage part, the pipette storage part 123, and the slide storage part 124 are provided in the storage space of the sliding door 120, the present invention is not limited thereto, and various inspection tools required for biopsy may be stored in the storage space of the sliding door 120.

The inspection module 200 according to an embodiment of the present invention may include an information recognition part 260 which is movable in X-axis, Y-axis, and Z-axis and is capable of recognizing the code 50 displayed on the inspection tool. The information recognition part 260 may be provided in the moving part 210. However, the present invention is not limited thereto, and the information recognition part 260 may be provided at various points as long as it can recognize the code 50 displayed on the inspection tool.

The inspection tool according to an embodiment of the present invention may be provided with a code 50. Specifically, the cord 50 may be provided in the first tube 11, the second tube 12, the slide 40, and the like.

The information recognition part 260 may scan and recognize the code 50. When the code 50 is scanned through the information recognition part 260, the information recognition part 260 may receive information about the inspection tool.

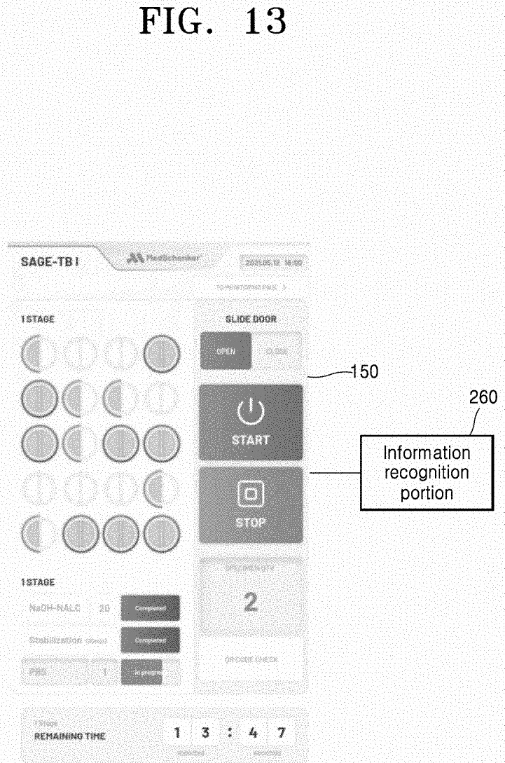

Referring to FIG. 13, the housing 110 according to an embodiment of the present invention may be provided with a display part 150 for displaying information recognized by the information recognition part 260 to the outside.

The display part 150 may be a display provided in the housing 110, and information recognized by the information recognition part 260 may be displayed on the display part 150. Specifically, when the code 50 of the inspection tool is scanned through the information recognition part 260, information about the inspection tool may be displayed on the display part 150.

The code 50 provided in the inspection tool according to an embodiment of the present disclosure may be a QR code. However, the present invention is not limited thereto, and the code 50 provided in the inspection tool may be any of various codes as long as the codes can provide information. The automatic preprocessing apparatus for biopsy according to an embodiment of the present invention may further include a waste processing part 130. The waste processing part 130 may automatically process the contaminated inspection tool.

If an inspector treats a contaminated inspection tool while an inspection is underway, there is a risk that the inspector will be infected by pathogens during the treatment process. In particular, since one side of the pipette 30 is open to the outside, there is a risk that the pipette 30 may be infected. The waste processing part 130 is provided to prevent the waste from being generated, and may process an inspection tool such as the pipette 30 through the waste processing part 130.

The waste processing part 130 may be provided in the housing 110, and the waste processing part 130 may be provided at a lower portion of the inspection module 200.

The waste processing part 130 may be provided with an outlet 131 communicating with an inner space of the housing 110, and the waste processing part 130 may be provided with a discharge bag 132 for storing an inspection tool.

Referring to FIGS. 4, 14, and 15, the outlet 131 may communicate with an inner space of the housing 110, and the discharge bag 132 may be provided at a lower portion of the outlet 131.

The moving part 210 of the inspection module 200 may move the inspection tool, and may move the inspection tool used through the moving part 210 to the outlet 131. When the inspection tool moved to the outlet 131 is dropped to the outlet 131, the inspection tool is moved to the discharge bag 132 through the outlet 131.

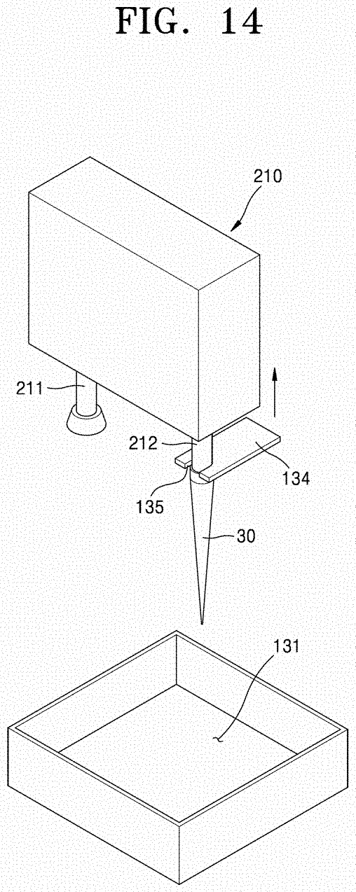

Referring to FIG. 14, the pipette 30 may be moved to the outlet 131 through the second moving part 212. According to an embodiment of the present invention, a separation part 134 having a groove 135 smaller than an upper diameter of the pipette 30 may be provided at an upper portion of the outlet 131.

The separation part 134 is provided to separate the pipette 30 from the second moving part 212, and the separation part 134 may be provided inside the housing 110. A process of separating the pipette 30 from the second moving part 212 through the separation part 134 is as follows.

The pipette 30 is disposed at the lower portion of the groove 135 through the second moving part 212. Thereafter, the pipette 30 is moved to an upper portion of the groove 135 through the second moving part 212. Since the groove 135 provided in the separation part 134 is smaller than the diameter of an upper portion of the pipette 30, the pipette 30 is caught by the separation part 134 when the pipette 30 is moved to the upper portion of the groove 135.

When the pipette 30 is caught by the separation part 134, the pipette 30 is separated from the second moving part 212 and is lowered to the outlet 131. The pipette 30 lowered to the outlet 131 is inserted into the discharge bag 132.

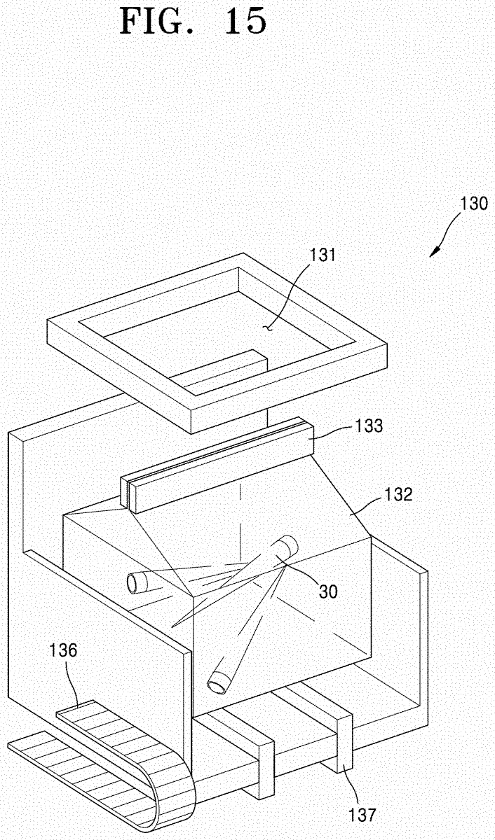

Referring to FIG. 15, the waste processing part 130 may include a sealing part 133 that may seal an upper portion of the discharge bag 132 in which the used inspection tool is stored. The sealing part 133 may seal the upper portion of the discharge bag 132 in which the inspection tool is stored.

As an upper portion of the discharge bag 132 in which the used inspection tool is stored is sealed by the sealing part 133, the inspection tool may be processed while preventing the contaminated material from being discharged to the outside.

According to an embodiment of the present invention, the housing 110 may be provided with a first opening/closing door 141 and a second opening/closing door 142. The first opening/closing door 141 is provided at a front surface of a position at which the inspection module 200 is disposed, and the second opening/closing door 142 is provided at a front surface of a position at which the waste processing part 130 is disposed.

The first opening/closing door 141 may be opened or closed. When the first opening/closing door 141 is opened, the inner space of the housing 110 provided with the inspection module 200 is communicated with the outside. The inspection module 200 may be maintained through the first opening/closing door 141.

The second opening/closing door 142 may be opened and closed. When the second opening/closing door 142 is opened, a space in which the waste processing part 130 is provided communicates with the outside. The discharge bag 132 provided in the waste processing part 130 may be removed through the second opening/closing door 142.

Referring to FIG. 15, the waste processing part 130 may include a moving part 136 to move the discharge bag 132 and an inclined part 137 to incline the discharge bag 132.

According to an embodiment of the present invention, the discharge bag 132 may be coupled to a frame, and the moving part 136 may move the frame to which the discharge bag 132 is coupled.

The inclined part 137 may adjust the inclination of the frame to which the discharge bag 132 is coupled, and the inclined part 137 may incline the frame to which the discharge bag 132 is coupled in the direction of the second opening/closing door 142.

As the position of the discharge bag 132 is changed through the moving part 136 and the inclined part 137 and the inclination of the discharge bag 132 is adjusted, an inspector may easily remove the discharge bag 132 from the waste processing part 130.

According to an embodiment of the present invention, the waste processing part 130 may be provided with an ultraviolet lamp (UV lamp) and an ion filter. The waste processing part 130 may be provided with the ultraviolet lamp to sterilize pathogens. In addition, since the ion filter is provided in the waste processing part 130, infection of pathogens may be minimized.

The automatic preprocessing apparatus for biopsy according to an embodiment of the present invention has the following effects.

The automatic preprocessing apparatus for biopsy according to an embodiment of the present invention has an advantage of automatically performing a biopsy while minimizing contamination of an inspection tool through an inspection module which performs a biopsy while automatically moving the inspection tool.

In addition, the automatic preprocessing apparatus for biopsy according to an embodiment of the present invention has an advantage that an inspection tool can be easily inserted into the housing through a sliding door sliding to the outside of the housing while the inspection tool is stored.

In addition, the automatic preprocessing apparatus for biopsy according to an embodiment of the present invention has an advantage in that the inspection tool can be processed while preventing the infection of an inspector by pathogens remaining in the inspection tool through a waste processing part capable of automatically collecting and sealing the inspection tool used for biopsy.

In addition, the automatic preprocessing apparatus for biopsy according to an embodiment of the present invention can reduce work time and improve work efficiency and improve accuracy of a biopsy as the inspection module automatically performs the biopsy.

The present invention relates to an automatic preprocessing apparatus for biopsy, which has an advantage of minimizing contamination of an inspection tool and automatically performing a biopsy through an inspection module for performing a biopsy while automatically moving the inspection tool.

In addition, the present invention has an advantage in that the inspection tool can be easily inserted into the housing through the sliding door sliding to the outside of the housing while storing the inspection tool.

In addition, the present invention has an advantage in that the inspection tool can be processed while preventing an inspector from being infected by pathogens remaining in the inspection tool through a waste processing part capable of automatically collecting and sealing the inspection tool used for biopsy.

In addition, the present invention can improve work efficiency while reducing work time as a biopsy is automatically performed through the inspection module, and can improve accuracy of the biopsy.

As described above, the present invention has been described with reference to the embodiment illustrated in the drawings, but this is merely exemplary, and those skilled in the art will understand that various modifications and embodiments may be made. Therefore, the real technical protection range of the present invention should be determined by the technical idea of the appended claims.

What is claimed is:

1. An automatic preprocessing apparatus for biopsy, comprising: a housing having an inner space; an inspection module provided in the inner space of the housing; and a sliding door provided with a storage space and slidably coupled to the housing so as to be inserted into the inner space of the housing or protrude to the outside of the housing, wherein the inspection module is movable in X-axis, Y-axis, and Z-axis directions and includes a moving part capable of gripping an inspection tool.

2. The automatic preprocessing apparatus for biopsy of claim 1, wherein the moving part comprises a first moving part capable of gripping the tube and a second moving part capable of gripping the pipette.

3. The automatic preprocessing apparatus for biopsy of claim 2, wherein the first moving part grips the cap of the tube through adsorption.

4. The automatic preprocessing apparatus for biopsy of claim 1, wherein the inspection module comprises a seating part on which the inspection tool is seated.

5. The automatic preprocessing apparatus for biopsy of claim 4, wherein the inspection module includes a cap adjustment part configured to remove the cap from an upper portion of the tube seated on the seating part or to couple the cap to the upper portion of the tube seated on the seating part.

6. The automatic preprocessing apparatus for biopsy of claim 5, wherein the cap adjustment part comprises: a plurality of contact parts which are slidable in a direction away from a side surface of the cap; and a rotation part to which the contact parts are coupled and which rotates the contact parts, wherein an elastic body is coupled to an end of the contact parts.

7. The automatic preprocessing apparatus for biopsy of claim 4, wherein the inspection module comprises a material injection part for injecting a material into the tube seated on the seating part.

8. The automatic preprocessing apparatus for biopsy of claim 1, wherein the inspection module comprises a mixing part that applies vibrations to the inspection tool or rotates the inspection tool.

9. The automatic preprocessing apparatus for biopsy of claim 1, wherein the inspection module comprises an information recognition part that is movable in X-axis, Y-axis, and Z-axis and is capable of recognizing a code displayed on an inspection tool.

10. The automatic preprocessing apparatus for biopsy of claim 9, wherein the housing is provided with a display part to externally display the information recognized by the information recognition part.

11. The automatic preprocessing apparatus for biopsy of claim 9, wherein the code formed in the inspection tool is a QR code.

12. The automatic preprocessing apparatus for biopsy of claim 1, wherein an inspection tool is stored in the storage space of the sliding door.

13. The automatic preprocessing apparatus for biopsy of claim 12, wherein the storage space of the sliding door is provided with at least one of tube storage parts in which a tube is stored, a pipette storage part in which a pipette is stored, and a slide storage part in which a slide for fixing a specimen is stored.

14. The automatic preprocessing apparatus for biopsy of claim 1, further comprising a waste processing part having an outlet communicating with an inner space of the housing and a discharge bag storing the inspection tool therein, wherein the moving part moves the used inspection tool to the discharge bag through the outlet.

15. The automatic preprocessing apparatus for biopsy of claim 14, wherein the waste processing part comprises a sealing part for sealing an upper portion of the discharge bag in which the used inspection tool is stored.

16. The automatic preprocessing apparatus for biopsy of claim 14, wherein the moving part comprises a second moving part capable of gripping the pipette, and a separation part provided at an upper portion of the outlet and having a groove smaller than an upper diameter of the pipette, and the second moving part disposes the pipette at a lower portion of the groove, and separates the pipette from the second moving part while moving the pipette upward, and lowers the pipette to the outlet.
